(19) United States
(12) Patent Application Publication
(10) Pub. No.: US 2025/0302661 A1
(43) Pub. Date: Oct. 2, 2025

(54) AN OSTOMY APPLIANCE AND A COUPLING THEREFORE

(71) Applicant: Coloplast A/S, Humlebaek (DK)

(72) Inventors: Mikael Andersen, Alleroed (DK); Jeppe Marckmann, Kastrup (DK)

(21) Appl. No.: 18/866,564

(22) PCT Filed: May 16, 2023
(86) PCT No.: PCT/DK2023/050121

§ 371 Date: Nov. 18, 2024

(30) Foreign Application Priority Data
May 18, 2022 (DK) PA202270263

(51) Int. Cl. A61F 5/448

(52) U.S. Cl. CPC A61F 5/448; A61F 2005/4486

(57) ABSTRACT

The disclosure relates to an ostomy appliance comprising: a first ring-shaped coupling part; a second ring-shaped coupling part; and a collecting bag in connection with the second coupling part. The first coupling part comprises an axial upright forming a ring collar. The second coupling part comprising an axial lock flange, an axial seal-supporting flange forming an inner seal-supporting surface, and a bulge sealing extending radially inward from the seal-supporting surface. The first coupling part and second coupling part are adapted to be detachably attached to each other in a coupled configuration such that the bulge sealing is positioned against the ring collar to provide a seal between the first and second coupling parts. The disclosure further relates to a coupling, a ring-shaped coupling part, a method for manufacturing a ring-shaped coupling part, and a method for attaching a collecting bag to a base plate of an ostomy appliance.

### TECHNICAL FIELD

The present disclosure relates to an ostomy appliance comprising a first coupling part, a second coupling part, and a collecting bag. The disclosure further related to a coupling for an ostomy appliance and a method for detachably attaching a collecting bag to a base plate of an ostomy appliance. Moreover, the disclosure relates to a method for manufacturing a coupling part of an ostomy appliance.

### BACKGROUND

In connection with surgery for a number of diseases in the gastro-intestinal tract, one of the potential consequences is that the patient is left with an abdominal stoma, such as a colostomy, an ileostomy, or a urostomy in the abdominal wall for the discharge of visceral contents. This discharge of visceral contents cannot be regulated at will. For that purpose, the user will have to rely on an ostomy appliance to collect the material emerging from such opening in a bag, which is later emptied and/or discarded at a suitable time.

The ostomy appliance may be a two-piece appliance comprising a base plate and a collecting bag which may be coupled to and un-coupled from each other through a coupling means. Such coupling means may for example be based on a first coupling part and a second coupling part. The base plate and one of the coupling parts may be attached, formed, or integrated as a single component, and similarly, the collecting bag and the other coupling part may be attached, formed, or integrated as a single component.

By having the ostomy appliance as a two-piece appliance, the base plate does not need to be separated from the skin of the user as often as exchange of the collecting bag requires. The base plate may need only to be changed every third or fourth day depending on the user, whereas the collecting bag may be changed more than once per day. Typically, it is desirable to need as few exchanges of the base plate as possible in order to reduce the risk of skin complications.

Connecting the two parts of an ostomy appliance coupling can be cumbersome for a user, both when it comes to correctly locating and aligning the two parts correctly and to ensuring a secure seal between the two coupling parts to prevent leakage of stomal fluids.

It is worth noting that users may potentially suffer from other health complications or handicaps.

Since the user typically exchanges the collecting several times daily, it is particularly desirable to provide ostomy appliances and couplings therefore which permit expeditious coupling and decoupling.

Users of ostomy appliances and health care professionals alike would welcome improvements in couplings for two-piece ostomy appliances.

### SUMMARY

On the above background, it is an object of examples of the disclosure to provide an ostomy appliance with improved capabilities of easily and securely performing coupling of the base plate and the collecting bag.

An aspect of the disclosure relates to an ostomy appliance comprising:
a first ring-shaped coupling part comprising an axial upright forming an outer contact surface having an outer contact surface diameter, the axial upright further forming a ring collar having an outer ring collar diameter exceeding the outer contact surface diameter;
a second ring-shaped coupling part comprising an axial lock flange forming an inner contact surface having an inner contact surface diameter less than the outer ring collar diameter, the second coupling part comprising an axial seal-supporting flange axially coextending the axial lock flange and forming an inner seal-supporting surface having an inner seal-supporting surface diameter exceeding the inner contact surface diameter, the second coupling part comprising a bulge sealing extending radially inward from the seal-supporting surface and terminating inwardly at an inner bulge sealing diameter less than the outer ring collar diameter;
and a collecting bag in connection with the second coupling part;
wherein the bulge sealing has a bulge sealing hardness and the ring collar has a ring collar hardness, wherein the bulge sealing hardness is less than the ring collar hardness,
wherein the first coupling part and second coupling part are adapted to be detachably attached to each other in a coupled configuration such that the bulge sealing is positioned against the ring collar to provide a seal between the first coupling part and the second coupling part.

The provision of an ostomy appliance with the above-described coupling parts can in general ensure an ostomy appliance in which coupling of the base plate and the collecting bag can be performed safely and quickly. In particular, since the inner contact surface diameter is less than the outer ring collar diameter, the coupling can provide an audible and/or tactile/haptic feedback as the second coupling part becomes correctly attached to the first coupling part. To perform this attachment, the user can apply force to the second coupling part to push it, in an axial direction, onto the first coupling part. Thereby, the lock flange can typically be pushed radially outwards by the ring collar until the entire lock flange has axially traversed this ring collar, upon which the lock flange snaps radially inwardly to lock the two coupling parts together.

Such an inward snap may provide audible and/or tactile/haptic feedback, e.g., a sound or a sensible light mechanical jolt or shock. More particularly, this feedback can for example be established by an edge of the lock flange snapping past the ring collar, by the inner contact surface of the lock flange striking the outer contact surface of the axial upright, by the ring collar striking the inner seal-supporting surface, or any combination thereof.

The provision of such audible and/or tactile/haptic feedback can provide a quick, reliable, and comprehensible response to the user, which permits the user to deduce that the two coupling parts have been correctly attached to each other. Without such feedback, the user may typically spend additional time to check and recheck that the two parts are correctly attached to each other, since an incorrect attachment is highly undesirable. A quick response is particularly desirable since users which may potentially suffer from poor dexterity or other health complications of handicaps, which can reduce the capability of the user to confirm whether two coupling parts are correctly coupled by other means such as visual confirmation.

In addition, the snap provided by lock flange and the ring collar can ensure that the coupled configuration of the two coupling parts is more clearly distinguishable from an uncoupled configuration. When attaching the two coupling parts, most of the relative motion of the two parts can be performed as a continuous gradual motion, but when the two parts are sufficiently close to the coupled configuration, they may typically snap together. This can for example occur when a sufficiently large fraction of the axial lock flange has been moved past the ring collar. Since the two parts can autonomously snap together when they are almost coupled, the risk of mistakenly leaving the two parts in an almost-coupled or incorrectly coupled configuration is reduced.

Moreover, the provision of a seal-supporting flange from which a bulge sealing extends inwardly can provide an improved seal between the two coupling parts. When the coupling parts are in the coupled configuration, the lock flange and the ring collar in combination ensure that the bulge sealing is axially pressed onto the ring collar. Moreover, the seal-supporting flange ensures that the bulge sealing is pressed radially inwardly onto the ring collar. Thus, the coupling of this disclosure ensures the seal between the bulge sealing and the ring collar is both based on axial and radial forces/contact, potentially improving the quality of the seal. Such an improved seal can further be enabled by the bulge sealing hardness being lower than the ring collar hardness, such that the bulge sealing is able to, at least partially, wrap around or envelop the ring collar.

Further, the provision of an axial seal-supporting surface from which a bulge sealing extends radially inwards ensures simplified manufacturing of an ostomy appliance, particularly simplified manufacturing of the second coupling part. A moulding process without needing a core mould part to be pulled radially inwardly after moulding the second coupling part is enabled. This improvement can be further complimented by a bulge sealing hardness being low, e.g., lower than the ring collar hardness. A bulge sealing with a low hardness can allow some flexibility/elasticity of the bulge sealing when removing an inner mould part.

Another aspect of the disclosure relates to a method for manufacturing a coupling part of an ostomy appliance, the method comprising the steps of:
assembling a first inner mould assembly comprising a first inner mould part and a second inner mould part;
moulding an intermediate coupling part comprising an axial lock flange forming an inner contact surface and comprising an axial seal-supporting flange being radially outwardly offset from the axial lock flange, wherein the first inner mould part defines the inner contact surface of the axial lock flange and the second inner mould part defines the outward radial offset of the seal-supporting flange relatively to the axial lock flange;
assembling a second inner mould assembly comprising a third inner mould part, wherein the intermediate coupling part is fixed to the second inner mould assembly; and
moulding a bulge sealing extending radially inward from the seal-supporting flange to produce the coupling part, wherein the third inner mould part and the intermediate coupling part collectively defines the volume in which the bulge sealing is moulded.

The above-exemplified manufacturing of a coupling part permits manufacturing of ostomy appliances as disclosed herein. Thereby, the manufacturing method can potentially ensure manufacturing an ostomy appliance coupling without the requirement of pulling a mould part radially inwardly during the moulding process. Further, one or more mould parts can potentially be re-used in the first and second inner mould assemblies.

Moreover, an aspect of the disclosure relates to a coupling for an ostomy appliance.

An additional aspect of the disclosure relates to a coupling part for an ostomy appliance.

Further, an aspect of the disclosure relates to a method for detachably attaching a collecting bag to a base plate of an ostomy appliance.

### BRIEF DESCRIPTION OF THE DRAWINGS

The accompanying drawings are included to provide a further understanding of embodiments and are incorporated in and constitute a part of this specification. The figures illustrate embodiments and together with the description explain principles of embodiments. Other embodiments and many of the intended advantages of all embodiments will be readily appreciated as they become better understood by reference to the following detailed description.

Various exemplary embodiments and details are described in the detailed description, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the effects or advantages shown. An effect or an advantage described in conjunction with a particular embodiment is not necessarily limited to that embodiment and may be practiced in other embodiments even if not so illustrated, or if not so explicitly described.

The figures can be listed as follows:

FIG. 1 illustrates schematic views of an ostomy appliance;

FIGS. 2a-d illustrate schematic views and cross-sectional views of first and second coupling parts;

FIG. 3 illustrates a schematic cross-sectional view of the first and the second coupling parts in a coupled configuration;

FIG. 4 illustrates a flow chart of method steps according to a method for detachably attaching a collecting bag to a base plate of an ostomy appliance;

FIGS. 5a-c illustrate perspective views of a second coupling part, a first coupling part, and a locking ring;

FIGS. 6a-b illustrate cut-out perspective cross-sectional views of a first coupling part and a second coupling part;

FIG. 7 illustrates a cross-sectional view of a second coupling part;

FIGS. 8a-b illustrate cross-sectional views of first and second coupling parts and various relevant diameters thereof; and

FIGS. 9a-f illustrate a manufacturing process for manufacturing a (second) ring-shaped coupling part of an ostomy appliance.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” etc., is used with reference to the orientation of the Figure(s) being described. Because components of embodiments can be positioned in a number of different orientations, the directional terminology is used for purposes of illustration and is not necessarily limiting. It is to be understood that other embodiments may be utilized, and structural or logical changes may be made without departing from the scope of the present invention. The following detailed description, therefore, is not to be taken in a limiting sense, and the scope of the present invention is defined by the appended claims.

It is to be understood that individual features of the various exemplary embodiments described herein may be combined with each other, unless specifically noted otherwise.

In this disclosure, solid and liquid wastes from the bowels may be referred to as both “output,” “waste(s),” and “fluids” interchangeably. A subject using the irrigation system may be referred to as “patient” or “user”. However, in some cases, “user” can also relate or refer to a health care professional (HCP), such as a doctor/physician, nurse, or other person.

In this disclosure, ‘axial’ or ‘axial direction’ should generally be considered as the direction along an axis extending through a passage defined (surrounded) by the ring-shaped elements of the coupling parts and through which passage stomal output can pass into the collecting bag. The radial direction should be understood as transverse to the axial direction. In some sentences, the words “inner” and “outer” can be used. These qualifiers should generally be perceived with reference to the radial direction, such that a reference to an “outer” element means that the element is farther away from a centre portion of a component than an element referenced as “inner”. In addition, “innermost” should be interpreted as the portion of a component forming a centre of the component and/or being adjacent to the centre of the component. In analogy, “outermost” should be interpreted as a portion of a component forming an outer edge or outer contour of a component and/or being adjacent to that outer edge or outer contour. In further analogy this is also the case with regard to the use of the words “uppermost” and “lowermost”.

Moreover, in this disclosure, “ring-shaped”, with reference to a coupling part, does not necessarily entail that all structural elements of a ring-shaped coupling part are necessarily perfectly circular around a central axis in the axial direction. A circular shape of structural elements may be preferable, since this permits coupling parts to be attached with different relative orientations. However, coupling rings according to this disclosure may also have structural elements which are not circular, potentially resulting in a reduced set of possible relative orientations of the coupling parts when they are attached. Further, the coupling parts may comprise cuts, protrusions, flanges, handgrips, loops/tabs for attachment of accessories etc, which are not necessarily circularly symmetrical around a central axis. Correspondingly, the term “diameter” should not be restricted to a radial dimension from centrum to periphery of a circle but may also include radial dimensions from a geometric centre to the periphery of non-circular rings.

The use of phrases such as (but not limited to) “substantially” and “approximately” as qualifiers of certain features or effects in this disclosure, is intended to simply mean that any deviations are within tolerances that would normally be expected by the skilled person in the relevant field.

The use of the word “generally” as a qualifier to certain features or effects in this disclosure is intended to mean—for a structural feature: that a majority or major portion of such feature exhibits the characteristic in question, and—for a functional feature or an effect: that a majority of outcomes involving the characteristic provide the effect, but that exceptionally some outcomes may not provide the effect.

The phrase “adjacent” should be interpreted as being “close to and/or in contact with, or temporarily brought into contact with, but not attached to [something].”

Regarding ostomy appliances according to this disclosure, the first coupling part comprises an axial upright forming an outer contact surface having an outer contact surface diameter. The axial upright further forms or comprises a ring collar having an outer ring collar diameter exceeding the outer contact surface diameter.

The first coupling part can be connected to or integrated with a base plate (of the ostomy appliance). Such a connection can for example be facilitated by a radially extending base flange connected to the axial upright at a (axially) distal end relatively to the ring collar. Such a base flange can also be referred to as a plate-connection flange. Attachment of the base flange to the base plate can be done by using one or more of a variety of suitable welding techniques, and/or by adhesion, and/or by mechanical fastening. In embodiments, the plate-connection flange can be connected to a backing layer of a base plate. The backing layer of an ostomy appliance backing layer typically comprises a polymeric material. A base flange can optionally contact the second coupling part via, e.g., the axial lock flange, thereby further improving rigidity of the first and second coupling parts in the coupled configuration.

The axial upright of the first coupling part includes an outer contact surface. In typical couplings, the axial upright extends 360 degrees around a passage-opening of the first coupling part, thereby generally providing the first coupling part with the ring-shape. The axial upright can be a flange or flange-like structure. The axial upright at least partly forms the ring-shaped first coupling part. In embodiments, the axial upright is perpendicular to the radial plate-connection flange. In embodiments, the axial upright extends at an angle to the radial plate-connection flange. In embodiments, the axial upright comprises at least a first portion extending at a first angle to the radial plate-connection flange, and at least a second portion extending at a second angle, different from the first angle, to the radial plate-connection flange. In embodiments, the outer contact surface of the axial upright extends at an angle to the radial plate-connection flange different from 90 degrees. In embodiments, an inner surface of the axial upright extends at an angle to the radial plate-connection flange different from 90 degrees. In embodiments, the outer contact surface extends at an angle making the surface inclined radially inward, i.e. towards a centre of the ring-shaped first coupling part, along its axial extent when viewed in relation to and beginning at the axial upright's connection to the radial plate-connection flange or, similarly, ending at the ring collar. In embodiments, the inner surface of the axial upright extends at an angle making the surface incline radially outward along its axial extent (when viewed in relation to and beginning at the axial upright's connection to the radial plate-connection flange).

The first coupling part includes a ring collar having an outer ring collar diameter exceeding the outer contact surface diameter. In embodiments, the ring collar forms an element of the first coupling part which is a separate element but connected to the axial upright. In embodiments, the ring collar is made from a material which is different from the material of a separate part of the axial upright. In embodiments, part of the axial upright and the radial plate-connection flange are made from the same material, for example such that they form a monolithic part of the coupling. In embodiments, the axial upright and the radial plate-connection flange are provided integrally with each other. In embodiments, the ring collar is made from a material which is different from the material the axial upright and, optionally, the radial plate-connection flange. In other embodiments, the ring collar is made from the same material as the axial upright. In embodiments, the ring collar is made from the same material as the axial upright and is provided integrally therewith. In embodiments, the ring collar, the axial upright, and the radial plate-connection flange are made from the same material. In embodiments, the ring collar, the axial upright, and the radial plate-connection flange are made from the same material and are provided integrally with each other.

In embodiments, wherein two or more elements of the first coupling part are of the same material and provided integrally with each other, the first coupling part is advantageously injection moulded in a “one-shot” injection moulding process. In embodiments, wherein at least two elements of the first coupling element are made from different materials, the two elements are advantageously injection moulded in a “two-shot” injection moulding process. In such a process, one of the elements is moulded first, after which the other element is moulded in direct contact with the first element. The second element can either be moulded before the material of the first element has set, to achieve a chemical bond between the two elements, or it can be moulded after the first element has set (typically by cooling off), in which case a mechanical fixing or keying arrangement can be used to join the two elements. In the case of further material differentiation between elements of the first coupling part, additional injection moulding steps (“shots”) can be applied. In addition, or alternatively, other methods and principles for joining the different materials of the first coupling part can be used, such as, but not limited to, welding and/or adhesion techniques where a separately produced component is joined by welding or adhesively to the first coupling part.

In embodiments, the ring collar of the first coupling part is made from a material which is particularly suitable for providing a tight seal with an element of the second coupling part. The seal should be at least liquid tight. In embodiments, the “tight seal” is both liquid- and gas-tight so that neither stomal output nor foul smelling gases can escape the ostomy appliance through the coupling.

In typical couplings according to this disclosure, the ring collar forms a distal portion of the first coupling part. The ring collar has an outer ring collar diameter exceeding the outer contact surface diameter. It thus extends radially outward beyond the outer contact surface of the axial upright. In other words, the ring collar can form a protrusion relatively to the axial upright, adapted to securely hold the second coupling part in place in a coupled configuration. In embodiments, the protrusion is suitably configured such that one or more elements of the second coupling part can easily slide over or past the protrusion.

The diameter of the ring collar can vary along the axial direction, for example due to a rounded shape of the ring collar. The “outer ring collar diameter” generally refers to the largest diameter of the ring collar along the axial direction. Generally, the ring collar has a greater, maximum width (radial direction) than a width of the axial upright.

The ring collar can include at least two portions, for example a first connection portion and a second seal-receiving portion. The first connection portion of the ring collar can generally be an axially extending flange or flange-like structure suitable for abutment and/or attachment with the lock flange of the second coupling part. The second seal-receiving portion of the ring collar can generally be provided as a radially extending portion suitable for establishing a seal in combination with the bulge sealing of the second coupling part.

The second coupling part comprises an axial lock flange forming an inner contact surface having an inner contact surface diameter. Relatively to the first coupling part, the inner contact surface diameter is less than the first outer ring collar diameter. Typically, the inner contact surface is adapted to be in abutment with the outer contact surface of the axial upright of the first coupling part, at least in the coupled configuration.

The second coupling part further comprises an axial seal-supporting flange axially coextending the axial lock flange and forming an inner seal-supporting surface. The seal-supporting flange axially coextends the lock flange in the sense that they may have approximately the same central axis around which they extend in the same direction of that axis. Further, the seal-supporting flange extends such that it is axially, at least partly displaced from the lock flange. The inner seal-supporting surface of the seal-supporting flange has a seal-supporting surface diameter exceeding the inner contact surface diameter, which ensures that the lock flange can lock to the ring collar of the first coupling part. That is, the seal-supporting surface is outwardly offset from the inner contact surface diameter, i.e., further away from the centre of the second coupling part than the inner contact surface diameter.

The lock flange can optionally terminate at an inner V-shaped lock flange corner position at the end of the lock flange which is closest to the bulge sealing. Here V-shaped should be interpreted in contrast to ‘rounded’ or ‘U-shaped’. Thus, a V-shaped corner does not relate to the angle of the corner, but indicates that the corner is a well-defined edge or intersection of two surfaces. This inner V-shaped lock flange corner can engage with the ring collar when the first coupling part and the second coupling part are in the coupled configuration, thereby potentially providing an improved fastening of the two coupling parts to each other. Further, the inner V-shaped lock flange corner may improve any audible and/or tactile/haptic feedback provided when attaching the two coupling parts in the coupled configuration.

Moreover, the second coupling part comprises a bulge sealing, extending radially inward from the seal-supporting surface and terminating inwardly at an inner bulge sealing diameter. This inner bulge sealing diameter is less than the outer ring collar diameter, which ensures that the bulge sealing is positioned against the ring collar to provide a seal between the first coupling part and the second coupling part.

The bulge sealing has a bulge sealing hardness and the ring collar has a ring collar hardness, and the bulge sealing hardness is typically less than the ring collar hardness. Further, the lock flange may have a lock flange hardness greater than the bulge sealing hardness, the seal-supporting flange may have a seal-supporting flange hardness greater than the bulge sealing hardness, and/or the axial outer contact surface may have an outer contact surface hardness greater than the bulge flange hardness.

Generally, hardness can be quantified using a durometer hardness test, for example according to an ASTM D2240-00 standard on a type OO scale. Having a soft bulge sealing can improve the seal of the coupling.

An ostomy appliance according to this disclosure further comprises a collecting bag in connection with the second coupling part. In principle, the collecting bag may be attached to the second coupling part in any manner, as long as the ostomy appliance can suitably provide passage of waste from the bowels of a user to the collecting bag via the coupling parts in a coupled configuration. The collecting bag may for example be attached to the seal-supporting flange, the lock flange, a dedicated bag-connection flange, or a combination thereof.

Typically, the first and the second coupling parts can be coupled to each other by gently pushing the second coupling part onto the first coupling part, whereby the axial lock flange contacts and axially slides past the ring collar, to a position where the axial lock flange locates at and abuts at least a portion of the outer contact surface of the axial upright. The axial lock flange may form a radially inward facing contact surface and has an inner contact surface diameter less than the outer contact surface diameter of the ring collar. The diameter of the inner contact surface may vary along the axial direction of the axial lock flange, but at least a minimum inner diameter is less than a maximum outer diameter of the ring collar.

In embodiments, the material of the axial lock flange of the second coupling part has sufficient flexibility to allow the second coupling part to be pushed onto and couple with the first coupling part, notwithstanding the axial lock flange having a smaller inner diameter than the outer diameter of the first coupling part. The lock flange is typically elastically deformed as it is pushed onto the first coupling part. In embodiments, one or more of the elements of the second coupling ring include one or more structures to provide flexibility of the ring-shaped second coupling ring for allowing smooth coupling with the first coupling ring. The lock flange can for example have one or more axially extending cuts, for example distributed around the second ring-shaped coupling part. Such cuts can improve flexibility of the lock flange, which ease coupling and decoupling.

Some ostomy appliances of the disclosure comprise a locking ring having an open state and a locked state. The locked state can, for example, have a smaller inner diameter than the open state. This locking ring can assist to lock and/or tighten the two coupling parts together to reduce the risk of unintentionally decoupling the two parts. It can for example be intended and configured to be positioned around the lock flange, such that when the locking ring is in its locked state, the lock flange cannot as easily be moved past the ring collar.

The locking ring can include means allowing to selectively shift the locking ring between an unlocked configuration (open state) and a locked configuration (locked state). By shifting the locking ring to the locked configuration, the inner diameter of the locking ring is decreased making the locking ring clamp against a radially outward facing surface of the axial lock flange, which in turn causes the radially inward facing lock contact surface of the axial lock flange to move into engagement with the outer contact surface of the axial upright of the first coupling part and thereby locking the ostomy appliance coupling in its coupled configuration.

The second coupling part can include a radially extending bag-connection flange and a radially extending proximal flange connected by the axial lock flange. The bag-connection flange can facilitate connection with the collecting bag and can be positioned, axially, between the axial lock flange and the seal-supporting flange. The proximal flange can be positioned at a distal end of the lock flange, relatively to the seal-supporting flange. The bag-connection flange, the lock flange, and the proximal flange can collectively form a ring groove for housing a locking ring. In embodiments, the groove (or “furrow”) can be generally U- or V-shaped in cross-section.

The bag-connection flange includes a proximal surface and a distal surface. In embodiments, the distal surface of the bag-connection flange is connected to the collecting bag of the ostomy appliance. Attaching the bag-connection flange to a collecting bag can be done by using one or more of a variety of suitable welding techniques, and/or by adhesion, and/or by mechanical fastening. In embodiments, the bag-connection flange can be connected to a welding- or reinforcement-material layer of the collecting bag, or directly to one of the polymeric laminate material layers typically forming the walls of a collecting bag of an ostomy appliance.

The proximal flange of the second coupling part can be adapted to be in abutment with a base flange of the first coupling part. The proximal flange includes a proximal surface and a distal surface. In the coupled configuration, the proximal surface of the proximal flange suitably abuts the base flange of the first coupling part, which provides a relatively firm foundation for the second coupling part during and after the action of coupling the two coupling parts.

In embodiments, the locking ring is provided in the groove of the second coupling part in its unlocked configuration from manufacture. In other embodiments, the locking ring can be provided separately from the other elements of the ostomy appliance coupling in an open state of the locking ring. The locking ring can be configured to allow a user to take the locking ring in the open state and apply it in the groove around the second coupling part, followed by manipulating the locking ring a closed state, yet still being in its unlocked configuration.

Generally, the outer contact surface diameter, outer ring collar diameter, the inner seal supporting surface diameter, and the bulge sealing diameter can for example be in the range of approximately 30-80 mm. Further, these diameters are typically within a few mm of each other, for example, the outer ring collar diameter may be approximately 1 mm larger than the outer contact surface diameter, the inner contact surface diameter, and the bulge inner bulge sealing diameter, and approximately 1 mm smaller than the seal-supporting surface diameter.

In the following, various embodiments are described with reference to the figures.

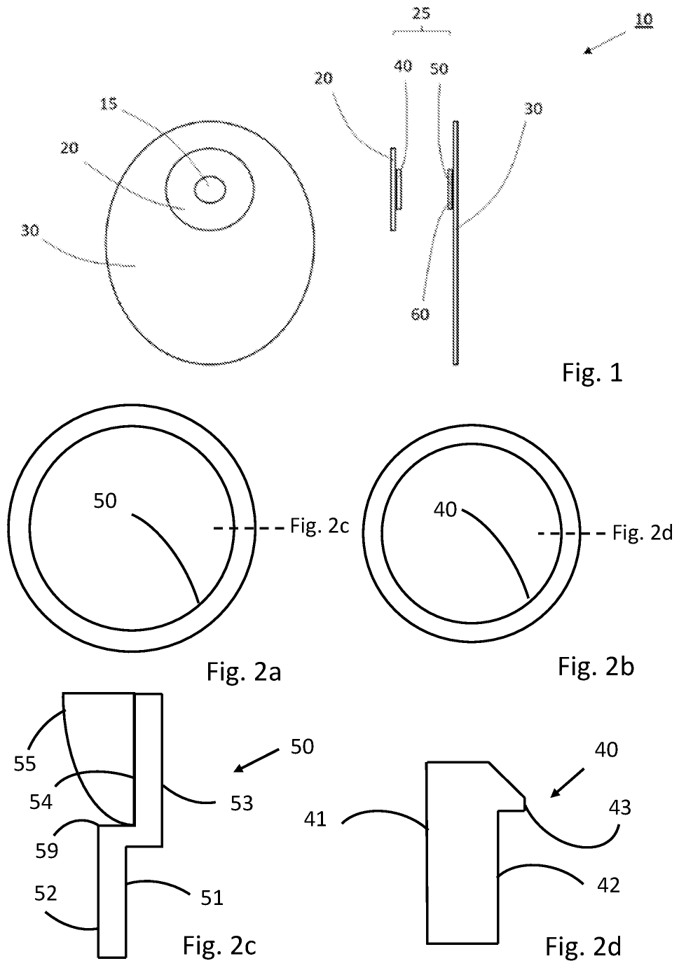

FIG. 1 illustrates schematic views of an ostomy appliance 10. The right part of the figure provides a side view of the ostomy appliance in which the two parts of the ostomy appliance 10 are decoupled. The left part of the figure provides a bottom view of the ostomy appliance 10 in which the two parts of the ostomy appliance are coupled. This view also corresponds to a view of the ostomy appliance 10 as seen from its attachment to a user/patient.

The ostomy appliance 10 comprises a base plate 20 attached to a first coupling part 40, and a collecting bag 30 attached to a second coupling part 50. Collectively, the first coupling part 40 and the second coupling part 50 provide a two-part coupling 25. These two coupling parts 40,50 can thus be detachably attached to each other to provide a sealed coupling between the collecting bag 30 and the base plate 20.

The base plate 20 and the first coupling part have a through-going stoma opening 15. The stoma opening 15 is configured to receive a stoma of the user and/or the stoma opening is configured to allow 15 output from the stoma to pass through the stoma opening 15 and into a collecting bag 30 attached to the base plate 20 via the two-part coupling 25. The stoma opening 15 is configured to allow passage of output from a proximal side of the base plate 20 to a distal side of the base plate and inside the collecting bag 30. Although the colleting bag 30 is illustrated as relatively flat, it is configured to permit expansion upon receiving waste.

FIGS. 2a-d illustrate schematic views and cross-sectional views of first and second coupling parts 40,50. Namely, FIG. 2a illustrates a top view of a second ring-shaped coupling part 50, FIG. 2b illustrates a top view of a first ring-shaped coupling part 40, FIG. 2c illustrates a cross-sectional view of the second coupling part 50, and FIG. 2d illustrates a cross-sectional view of the first coupling part FIG. 2d. The two coupling parts 40,50 are illustrated separately and uncoupled.

The top views in FIGS. 2a-b of the first and second coupling parts 40,50 clearly show the ring-shape of the two parts 40,50. The central hole of each the ring shapes serve as stoma openings. That is, when the two parts 40,50 are coupled in an ostomy appliance, the central hole in each ring can collectively allow output from the stoma of a user to pass into a collecting bag.

Both FIG. 2a and FIG. 2b show dashed lines with reference to FIG. 2c and FIG. 2d, respectively, which indicate the planes in which the cross-sectional views in FIG. 2c and FIG. 2d are provided.

FIG. 2c illustrates a cross-sectional view of the second coupling part 50. This coupling part comprises an axial lock flange 51 and an axial seal-supporting flange 53 axially coextending the lock flange 54. In this example, the axial lock flange 51 and the seal-supporting flange 53 are moulded as a single piece from a material comprising a polyolefin, such as a low-density polyethylene, or other suitable thermoplastics. They are connected by a radially outwardly extending connection positioned axially between the axial lock flange 51 and the seal-supporting flange 53.

The axial lock flange 51 has an inner contact surface 52 having an inner contact surface diameter, and the seal-supporting flange 53 has an inner seal-supporting surface 54. The diameter of the inner contact surface 52 is less than the diameter of the seal-supporting surface 54. In other words, the seal-supporting flange 53 and the seal-supporting surface 54 is positioned radially outwardly relatively to the lock flange 51 and the inner contact surface 52.

The illustrated lock flange terminates at an inner V-shaped lock flange corner 59. This lock flange corner 59 is located at the end of the lock flange closest to the bulge sealing.

The second coupling part 50 further comprises a bulge sealing 55 extending radially inward from the seal-supporting surface 54. It terminates at an inner bulge sealing diameter which is less than the diameter of the inner contact surface 52. In this example, it is made from a material comprising a thermoplastic elastomer.

FIG. 2d illustrates a cross-sectional view of the first coupling part 40. This coupling part 40 comprises an axial upright forming a ring collar 43. The axial upright 41 further forms an outer contact surface 42. The outer diameter of the ring collar 43 exceeds the diameter of the outer contact surface 42. The ring collar 43 thus forms an outer protrusion relatively to the outer contact surface. In this example, the ring collar 43 and the rest of the axial upright 41 is moulded as a single piece from a material low-density polyethylene.

In addition, the outer diameter of the ring collar exceeds the diameter of the inner contact surface 52 of the second coupling part but is smaller than the diameter of the inner seal-supporting surface.

Based on the materials used for moulding the pieces, the bulge sealing has a bulge sealing hardness which is less than the (material) hardness of the axial upright 41, of the lock flange 51, and of the seal-supporting flange 53.

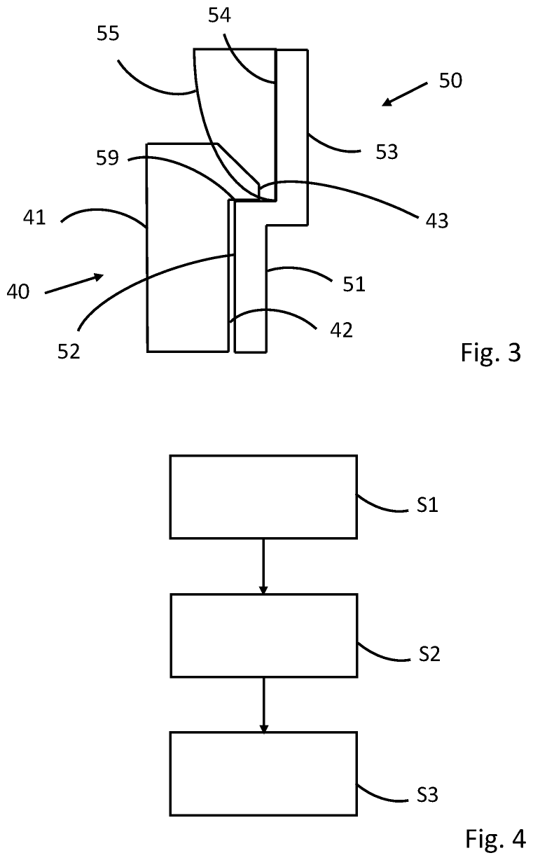

FIG. 3 illustrates a schematic cross-sectional view of the first and the second coupling parts 40,50 in a coupled configuration. The first and second coupling parts 40,50 of this illustration are similar to those illustrated in FIGS. 2a-d. Further, the plane of the cross-sectional view is similar. However, in FIG. 3, the two coupling parts 40,50 are illustrated in a coupled configuration. Here, the two parts 40,50 have been pressed together by, e.g., a user. More specifically, the second coupling part 50 has been pushed, axially, downwardly (relatively to the illustration) onto the first coupling part 40. In this process, the lock flange 51 is elastically bent outwardly by the ring collar 43 until the entire lock flange 51 has been pressed past the ring collar 43, upon which the lock flange 51 has snapped inwardly, such that the outer contact surface 42 and the inner contact surface 52 are at contact or are positioned closely together.

The inner V-shaped lock flange corner 59 of the second coupling part 50 engages with the ring collar 43 of the first coupling part 40, thereby providing a firm fixture of the two coupling parts 40,50 to each other.

In this coupled configuration, the bulge sealing 55 is positioned against the ring collar 43 to provide a seal between the first coupling part 40 and the second coupling part 50. In the illustration, the bulge sealing 55 spatially overlaps with the axial upright 41 and the ring collar 43. However, in practice, the bulge sealing 55 is deformed, due to its relatively lower hardness. This deformation provides an advantageous seal between the two parts 40,50.

FIG. 4 illustrates a flow chart of method steps according to a method for detachably attaching a collecting bag to a base plate of an ostomy appliance.

In a first method step S1, a first ring-shaped coupling part is provided. The first coupling part comprises an axial upright forming an outer contact surface having an outer contact surface diameter, the axial upright further forming a ring collar having an outer ring collar diameter exceeding the outer surface diameter. The first coupling part can be attached to or integrated with the base plate.

In a next method step S2, a second ring-shaped coupling part is provided. The second coupling part comprises an axial lock flange forming an inner contact surface having an inner contact surface diameter less than the outer ring collar diameter. In addition, the second coupling par comprises an axial seal-supporting flange axially coextending the axial lock flange and forming and inner seal-supporting surface. The second coupling part further comprises a bulge sealing extending radially inward from the seal-supporting surface and terminating inwardly at an inner bulge sealing diameter less than the outer ring collar diameter. Moreover, the second coupling part is in connecting with the collecting bag. The bulge sealing has a bulge sealing hardness and the ring collar has a ring collar hardness, wherein the bulge sealing hardness is less than the ring collar hardness.

In a next method step S3, the second coupling part is pressed onto the first coupling part. The two parts are pressed together such that, just prior to contact between the first and second coupling parts, the axial lock flange is located next to the ring collar (as opposed to the seal-supporting surface being located next to the ring collar). The two parts are pressed towards each other such that the axial lock flange is moved across the ring collar. Due to the ring collar diameter exceeding the inner contact surface diameter, the lock flange is at least partly pushed radially outwards by the ring collar as the axial lock flange is moved across the ring collar. Optionally, the lock flange may be provided with axially oriented cuts around the ring shape of the lock flange to improve flexibility of the lock flange.

Since the inner contact surface diameter is smaller than the ring collar diameter and the seal-supporting surface diameter is larger than the ring collar diameter, the two coupling parts collectively provide a click when the (entire) axial lock flange has moved across the ring collar. This click may, more specifically, be established by an edge of the lock flange snapping past the ring collar, by the inner contact surface of the lock flange striking the outer contact surface of the axial upright, by the ring collar striking the inner seal-supporting surface, or by any combination thereof. The click may be audible, i.e. the click may constitute audible feedback, which a user is able to hear. Alternatively or additionally, the click may constitute tactile/haptic feedback, e.g. a light mechanical jolt or shock, which a user can easily comprehend.

Preferably, the two coupling parts are in the coupled configuration when the entire lock flange or the entire inner contact surface has been axially moved across the ring collar. In the coupled configuration, the ring collar is positioned such that it slightly deforms the bulge sealing, and such that the bulge sealing at least partially surrounds the ring collar. Thereby, the second coupling part has been pressed onto the first coupling part such that the bulge sealing is positioned against the ring collar to provide a seal between the first coupling part and the second coupling part.

Further, the second coupling part is pressed onto the first coupling part such that the bulge sealing is positioned against the ring collar to provide a seal between the first coupling part and the second coupling part (when the two coupling parts are fully attached in a coupled configuration).

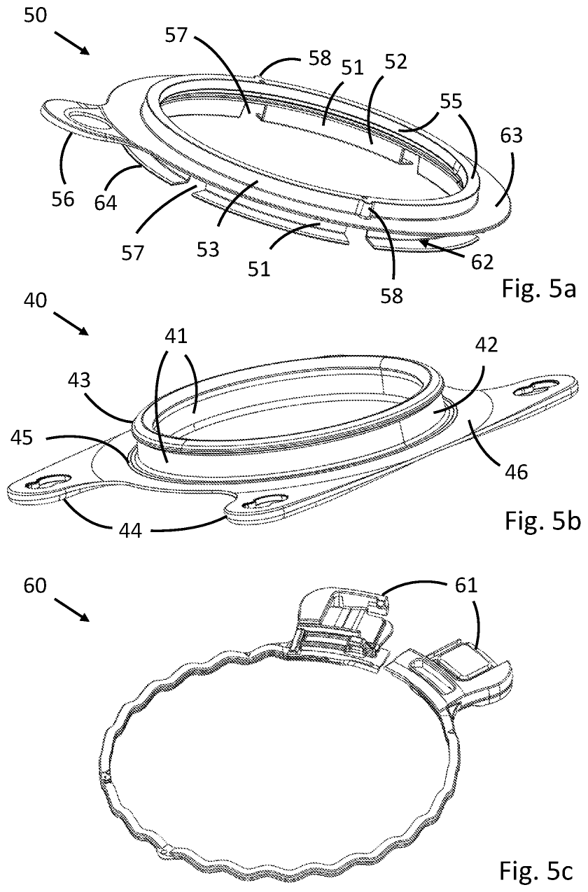

FIGS. 5a-c illustrate perspective views of a second coupling part 50, a first coupling part 40, and a locking ring 60.

In FIG. 5a, a second coupling part 50 is illustrated. In addition to an axial lock flange 51 and an axial seal-supporting flange 53 axially coextending therefrom, the coupling part 50 has additional features.

A radially extending bag-connection flange 63 is located axially between the lock flange 51 and the seal-supporting flange 53. When the coupling 40,50,60 is integrated in an ostomy appliance, the collecting bag is connected to the second coupling part via this bag-connection flange 63.

At the lower axial end of the axial lock flange 51, at the distal end of the lock flange 51 relatively to the seal-supporting flange 53 and the bag-connection flange 63, a radially extending proximal flange 64 is located. The proximal flange 64, the lock flange 51, and the bag-connection flange 63 collectively form a ring groove 62. This ring groove 62 is configured to house or host a locking ring 60 as the one illustrated in FIG. 5c.

The second coupling part 50 further comprises several axially extending cuts 57 in the lock flange 51 and in the proximal flange 64. These improve the flexibility of the lock flange 51, thereby easing coupling and decoupling the first and second coupling parts 40,50 to each other.

Moreover, the coupling part 50 comprises a radially extending detachment grip 56 which provides a grip which a user can pull to detach the second coupling part 50 from the first coupling part 40. By pulling the grip 56 axially away from the lock flange 51, towards the seal-supporting flange 53, the lock flange 51 can release its fastening locally near the position of the grip 56, thereby allowing easy detachment of the second coupling part 50 from the first coupling part 40.

The second coupling part 50 further comprises two local protrusions 58, which extend radially outwardly from the axial seal-supporting flange locally around the coupling part 50 and extending axially from the bag connecting flange 63. Integrating such local protrusions enable easier manufacturing of the second coupling part. Particularly, these local protrusions can serve as entry points of the material of the bulge sealing 55 during direct injection moulding of the bulge sealing 55. The choice of location of the local protrusions 58 does not interfere with the attachment and detachment of the two coupling parts with each other. When the coupling is integrated in an ostomy appliance, these local protrusions 58 can further be hidden inside the collecting bag.

In FIG. 5b, a first coupling part 40 is illustrated. In addition to the axial upright 41 forming the ring collar 43, this coupling part 40 further comprises a base flange 45 connected to the axial upright at the distal end relatively to the ring collar and extending radially outwardly. The base flange 45 is attached to a base plate 46. In addition, the first coupling part 40 comprises fastening loops 44 extending radially from the base plate. These loops can, for example, permit attachment of the coupling part 40 to the user, or of auxiliary items to the first coupling part 40.

FIG. 5c illustrates a locking ring 60 having a circular shape and comprising a locking ring clamping mechanism 61. The clamping mechanism allows a user to mechanically switch the locking ring 60 between an open state and a locked state. In this illustration, the clamping mechanism 61 is disassembled.

The locking ring 60 is sized to fit into the ring groove 62 of the second coupling part 62. When the second coupling part 50 is pressed onto the first coupling part 40 such that the bulge sealing 55 is positioned against the ring collar 43, the locking ring 60 is mechanically switched from the open state to the locked state. In the locked state, the locking ring 60 applies radial inward pressure to the lock flange 51, such that the inner contact surface 52 of the second coupling part 50 is pressed onto the outer contact surface 41 of the first coupling part 40. This reduces the risk of the two coupling parts 40,50 being unintentionally detached, since the freedom of movement of the lock flange 51 is reduced by the locking ring 60 in the locked state, such that the lock flange 51 is restricted from being radially deformed allowing movement past the ring collar 43.

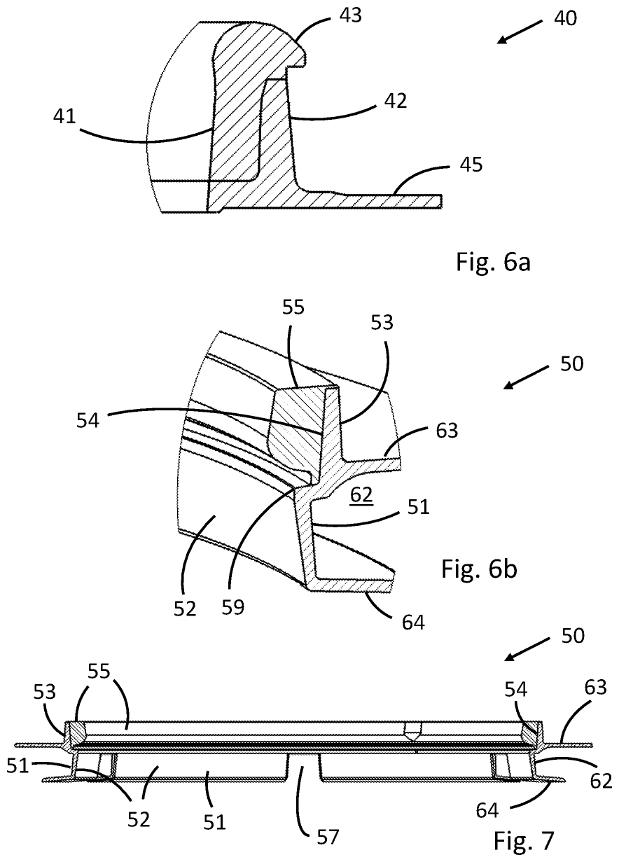

FIG. 6a-b illustrate cut-out perspective cross-sectional views of a first coupling part 40 and a second coupling part 50.

In the example of a first coupling part illustrated in FIG. 6a, the first coupling part is moulded in two different materials. In particular, the ring collar 43 is made from a material comprising a polyolefin elastomer. Thereby, an improved seal can be provided by the combination of materials of the bulge sealing 55 and the ring collar 43. Particularly, the bulge sealing may be relatively soft, whereas the ring collar is relatively hard. Further, the ring collar 43 has a greater hardness than the other parts of the first locking part, in particular the base-connecting flange 45, which has some flexibility to permit a proper fit of the coupling part 40 (and the base plate) to the curvature of the body of the user.

In the illustrated first coupling part, the outer contact surface 42 has a slight angle relatively to a central axis. The outer contact surface 42 inclines inwardly in an axial direction towards the ring collar 43 at an angle less than 15 degrees. This inclination can ensure that the locking flange 51 is pressed towards the ring collar 43 in the coupled configuration.

In FIG. 6b, an additional example of a second coupling part 50 is provided. Here, the inner contact surface 52 has a slight angle relatively to a central axis. The inner contact surface 52 inclines inwardly in an axial direction towards the seal-supporting flange 53 at an angle less than 15 degrees. This inclination can ensure easer attachment of the second coupling part 50 onto the first coupling part 40, as the area of the opening of the second coupling part at the bottom of the axial lock flange 51 is larger, allowing easer insertion of the first coupling part 40 therein.

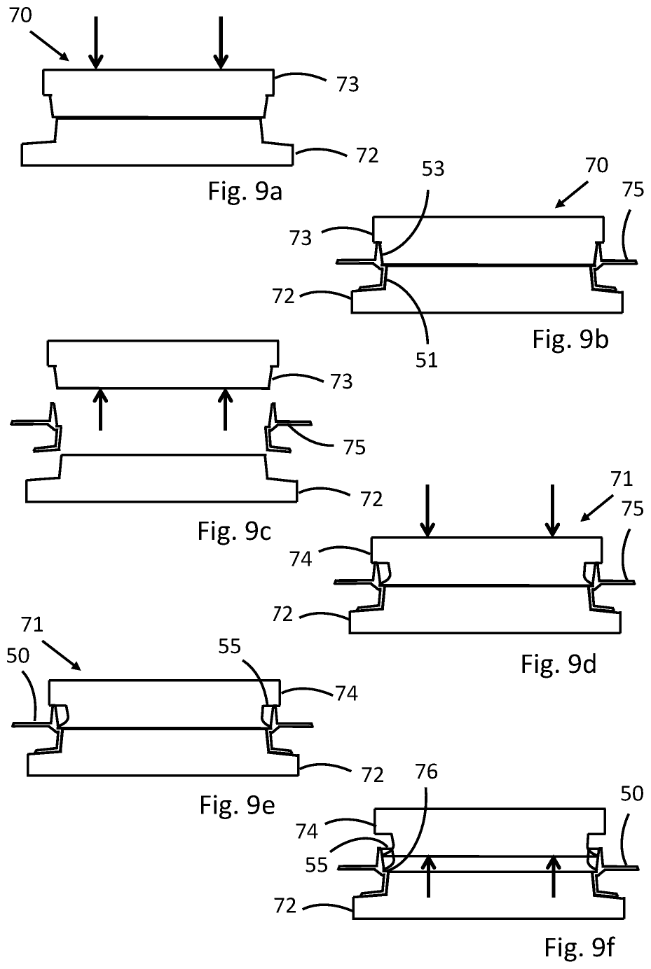

Furthermore, the inner seal-supporting surface 54 has a slight angle relatively to a central axis. The seal-supporting surface 54 inclines outwardly in an axial direction away from the lock flange 51 at an angle less than 15 degrees. Thereby, the seal-supporting surface supports the bulge sealing 55 adequately, while allowing easier removal of inner mould parts as explained in relation to FIGS. 9a-f.

The bulge sealing 55 is shaped such that when the first and the second coupling parts are attached to each other in a coupled configuration, the bulge sealing 55 axially extends across the (radially) outermost part of the bulge sealing. Thereby, an improved seal between the first and second coupling parts 40,50 is provided.

FIG. 7 illustrates a cross-sectional view of a second coupling part 50. The illustrated coupling part 50 is similar to the coupling part illustrated in FIG. 5a, but the cross-sectional view provides additional insight to the mechanical design of the coupling part 50.

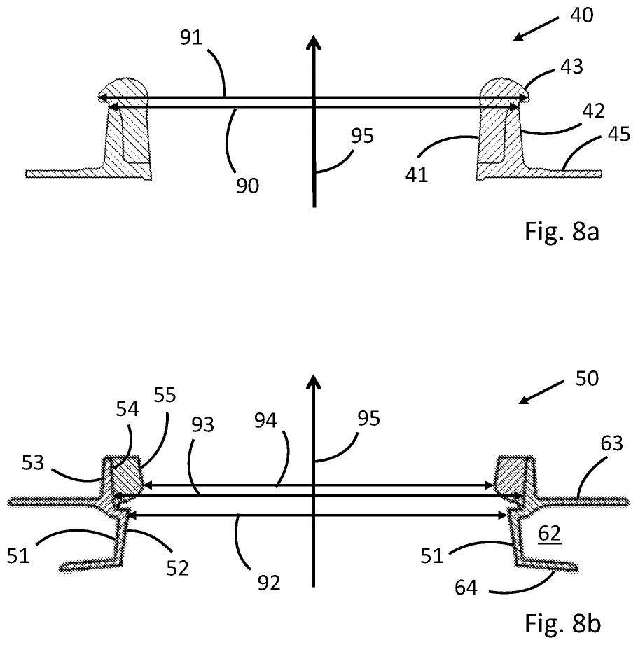

FIG. 8a-b illustrate cross-sectional views of first and second coupling parts and various relevant diameters 90-94 thereof. Moreover, each of the two figures, FIG. 8a and FIG. 8b, illustrates a central axis 95 around which each coupling parts 40,50 may extend circularly.

FIG. 8a illustrates that the outer ring collar diameter 91 may be measured at the largest diameter of the ring collar (relatively to the central axis 95). The outer contact surface diameter 90 may be measured at the portion of the outer contact surface 90 which is nearest the ring collar 43 (relatively to the central axis 95). Alternatively, the outer contact surface diameter 90 may be measured at the smallest diameter of the outer contact surface.

FIG. 8b illustrates that the inner bulge sealing diameter 94 may be measured at the furthest inward point of the bulge sealing 55 (relatively to the central axis 95). The inner contact surface diameter 92 may be measured at the furthest inward point of the inner contact surface 52 (relatively to the central axis 95). Alternatively, inner contact surface diameter 92 may be measured at the end of the inner contact surface 52 nearest the seal-supporting flange 53. The inner seal-supporting surface diameter 93 may be measured at the furthest inward point of the inner contact surface 54 (relatively to the central axis 95). Alternatively, it may be measured at the end of the inner seal-supporting surface 54 which is nearest the lock flange 51.

The central axes 95 illustrated in FIGS. 8a and 8b are further useful for displaying the meaning of ‘axial’ and ‘axial direction’, which correspond to directions substantially perpendicular to the central axes 95. The terms ‘radial’ or ‘radial direction’ correspond to directions substantially perpendicular to the central axes 95.

FIGS. 9a-f illustrate a manufacturing process for manufacturing a (second) ring-shaped coupling part 50 of an ostomy appliance. Each of the subfigures provide a cross-sectional side view mould parts and the (intermediate) coupling part during the process.

In FIG. 9a, a first inner mould assembly 70 is assembled. This mould assembly 70 comprises a first inner mould part 72 and a second inner mould part 73. The inner mould assembly 70 has been assembled by bringing the two moulding parts 72,73 together, as indicated by arrows.

In FIG. 9b, an intermediate coupling part 75 is moulded. The intermediate coupling part 75 is an intermediate product produced towards manufacturing the final product, i.e. the coupling part. In addition to the inner mould assembly, one or more outer mould parts are also used (but not shown), which determine the outer surface shape of the intermediate coupling part. Collectively, the inner mould assembly and any outer mould parts defines the volume in which the intermediate coupling part is moulded.

The intermediate coupling part comprises an axial lock flange 51 forming an inner contact surface having an inner contact surface diameter. Further, the intermediate coupling part comprises an axial seal-supporting flange 53 axially coextending the axial lock flange 51 and forming an inner seal-supporting surface, which has an inner seal-supporting surface diameter exceeding the inner contact surface diameter.

The outer shape of the inner mould assembly 70 determines the inner surface of an intermediate coupling part 75. In particular, the first inner mould part 72 defines the inner contact surface, and the second inner mould part 73 defines the inner seal-supporting surface.

Typically, injection moulding is employed for moulding the intermediate coupling part 75. Typically, a material comprising a low-density polyethylene is used. Further, the manufacturing process typically involves some curing time after moulding and before proceeding to the next manufacturing step.

In FIG. 9c, the first mould assembly 70 is disassembled by removing the second inner mould 73 part from the first inner mould part 72. The intermediate coupling part 75 can thereby be released, at least temporarily.

In FIG. 9d, a second mould assembly 71 is assembled by bringing a third inner mould part 74 together with the first inner mould part 72. These two mould parts 72,74, and thereby the second inner mould assembly 71, further fix the intermediate coupling part 75.

In FIG. 9e, a bulge sealing 55 is moulded. Optionally, one or more outer mould parts can be used, but depending on the specific shape of the second inner mould assembly 71 and the intermediate coupling part 75, such outer mould parts may not be necessary, as is the case in the illustrated example of a manufacturing process. The third inner mould part 74 and the intermediate coupling part 75 collectively define the volume in which the bulge sealing 55 is moulded. And in this example, only the third inner mould part 74 and the intermediate coupling part 75 defines the volume in which the bulge sealing 55 is moulded.

The step of moulding the bulge sealing 55 thereby concludes the production of the (second) coupling part 50.

The moulded bulge sealing 55 extends radially inward from the seal-supporting surface and terminates inwardly at an inner bulge sealing diameter less than the inner contact surface diameter.

Again, injection moulding is typically employed for moulding the bulge sealing. Typically, a material comprising a thermoplastic elastomer is used. Further, as previously, the manufacturing process typically involves some curing time after moulding.

Preferably, the resulting bulge sealing 55 is softer than the axial lock flange and the seal-supporting flange. This can, for example, be achieved by moulding the intermediate coupling part with a first moulding material and the bulge sealing with a second moulding material different from the first moulding material. Thereby, the bulge sealing can be manufactured with a bulge sealing hardness, and the axial lock flange (and the seal-supporting flange) can be manufactured with an axial lock flange hardness, in which the bulge sealing hardness is less than the axial lock flange hardness.

In FIG. 9f, the second mould assembly 71 is disassembled by moving the third inner mould 74 part away from the first inner mould part 72. In this context, note that the coupling part 50 is manufactured with an inner undercut 76. More specifically, the bulge sealing 55 forms an inner undercut 76, i.e., the inner undercut 76 would not be present if the bulge sealing 55 was not present. If the entire coupling part 50 (including the bulge sealing 55) was manufactured using a sufficiently hard material, the presence of this inner undercut 76 would prevent a simple withdrawal of the third inner mould part 74 to disassemble the second inner mould assembly 71. Instead, an inner pull or other complicating manufacturing procedures would be required.

However, due to the softness of the bulge sealing 55, and the geometry of the rest of the coupling part 50, particularly the seal-supporting surface 53, the third inner mould part 74 can simply be removed since the shape of the bulge sealing 55 deflects/deforms due to the elasticity/softness of the bulge sealing 55.

In the figure, FIG. 9f, the bulge sealing 55 is illustrated without any deflection or deformation to show that, during disassembly, the third inner mould part 74 overlaps spatially with the volume which the bulge sealing 55 occupies when it is not deflected. However, in practise, the bulge sealing 55 is indeed deformed, thus permitting passage of the third inner mould part 74.

As a further step of the manufacturing method, a collecting bag can be attached to the coupling part 50, for example via a bag-connection flange of the coupling part 50.

In the following, various embodiments are described with reference to the figures.

In some ostomy appliances according to this disclosure, the outer contact surface radially contacts the inner contact surface when the first coupling part and the second coupling part are attached to each other in the coupled configuration.

In some ostomy appliances according to this disclosure, the axial lock flange has an axial lock flange hardness, wherein the bulge sealing hardness is less than the axial lock flange hardness.

In some ostomy appliances according to this disclosure, the bulge sealing hardness is based on a material hardness of the bulge sealing, wherein the material hardness is at most 55, for example at most 50, for example at most 45, such as at most 40 when measured according to an ASTM D2240-00 standard on a type OO scale.

In some ostomy appliances according to this disclosure, the bulge sealing is made from a material comprising a thermoplastic elastomer.

In some ostomy appliances according to this disclosure, the axial lock flange, the axial upright, the seal-supporting flange, or any combination thereof are made from a material comprising a low-density polyethylene.

In some ostomy appliances according to this disclosure, the ring collar is made from a material comprising a polyolefin elastomer.

In some ostomy appliances according to this disclosure, the second coupling part comprises a radially extending bag-connection flange axially positioned between the axial lock flange and the seal-supporting flange, wherein the collecting bag is connected to second coupling part via the bag-connection flange.

In some ostomy appliances according to this disclosure, the ostomy appliance further comprises a locking ring having an open state and a locked state, wherein the second coupling part comprises a radially extending proximal flange connected to the axial lock flange at a distal end relatively to the seal-supporting flange such that the bag-connection flange, the axial lock flange, and the proximal flange collectively form a ring groove for housing the locking ring, wherein the locking ring locks the second coupling part to the first coupling part when the first coupling part and the second coupling part are attached to each other in the coupled configuration and when the locking ring is housed in the ring groove in the locked state.

In some ostomy appliances according to this disclosure, the first coupling part comprises a radially extending base flange connected to the axial upright at a distal end relatively to the ring collar, wherein the base flange axially contacts the second coupling part when the first coupling part and the second coupling part are attached to each other in the coupled configuration.

In some ostomy appliances according to this disclosure, the second coupling part comprises one or more local protrusions extending radially outwardly from the axial seal-supporting flange locally around the second ring-shaped coupling part and extending axially from the bag-connection flange.

In some ostomy appliances according to this disclosure, the inner seal-supporting surface is radially outwardly offset from the outer ring collar diameter.

In some ostomy appliances according to this disclosure, the bulge sealing axially extends across the outermost part of the ring collar when the first coupling part and the second coupling part are attached to each other in the coupled configuration.

In some ostomy appliances according to this disclosure, the bulge sealing forms an inner undercut of the second coupling part.

In some ostomy appliances according to this disclosure, the entire inner seal-supporting surface is outwardly offset from the inner contact surface of the axial lock flange.

In some ostomy appliances according to this disclosure, wherein the inner seal-supporting surface has a non-decreasing diameter along its entire axial direction away from the axial lock flange.

In some ostomy appliances according to this disclosure, the inner bulge sealing diameter is less than the inner contact surface diameter.

In some ostomy appliances according to this disclosure, the lock flange comprises one or more axially extending cuts.

In some ostomy appliances according to this disclosure, the base flange has a base flange hardness which is less than the ring collar hardness.

In some ostomy appliances according to this disclosure, the lock flange terminates axially at an inner V-shaped lock flange corner located at an end of the lock flange closest to the bulge sealing, wherein the lock flange corner engages with the ring collar when the first coupling part and the second coupling part are in the coupled configuration.

In some manufacturing methods according to this disclosure, the bulge sealing forms an inner undercut of the coupling part, wherein the method further comprises a step of axially displacing the third inner mould part to disassemble the second inner mould assembly and release the coupling part, wherein the step of axially displacing the third inner mould part is involves deforming the bulge sealing.

In some manufacturing methods according to this disclosure, the intermediate coupling part is moulded using a first moulding material and the bulge sealing is moulded using a second moulding material, such that the bulge sealing has a bulge sealing hardness and the axial lock flange has an axial lock flange hardness, wherein the bulge sealing hardness is less than the axial lock flange hardness.

In some manufacturing methods according to this disclosure, the second inner mould assembly comprises the first inner moulding part.

In some manufacturing methods according to this disclosure, the method further comprises a step of connecting a collecting bag to the coupling part.

An additional aspect of the disclosure relates to a method for detachably attaching a collecting bag to a base plate of an ostomy appliance, the method comprising the steps of:
providing a first ring-shaped coupling part comprising an axial upright forming an outer contact surface having an outer contact surface diameter, the axial upright further forming a ring collar having an outer ring collar diameter exceeding the outer surface diameter;
providing a second ring-shaped coupling part comprising an axial lock flange forming an inner contact surface having an inner contact surface diameter less than the outer ring collar diameter, the second coupling part comprising an axial seal-supporting flange axially coextending the axial lock flange and forming an inner seal-supporting surface having an inner seal-supporting surface diameter exceeding the inner contact surface diameter, the second coupling part comprising a bulge sealing extending radially inward from the seal-supporting surface and terminating inwardly at an inner bulge sealing diameter less than the outer ring collar diameter, wherein the second coupling part is in connection with the collecting bag, wherein the bulge sealing has a bulge sealing hardness and the ring collar has a ring collar hardness, wherein the bulge sealing hardness is less than the ring collar hardness; and
pressing the second coupling part onto the first coupling part such that the axial lock flange is moved across the ring collar and such that the bulge sealing is positioned against the ring collar to provide a seal between the first coupling part and the second coupling part.

In some methods for detachably attaching a collecting bag to a base plate according to this disclosure, the first coupling part and the second coupling part collectively provides a click when the axial lock flange has moved across the ring collar.

Method steps implicit in view of the ostomy appliance and method of manufacturing the ring-shaped coupling may optionally apply to the method of detachably attaching the collecting bag to a base plate of an ostomy appliance.

### LIST OF REFERENCES

10 ostomy appliance
15 stoma opening
20 base plate
25 two-part coupling
30 collecting bag
40 first coupling part
41 axial upright
42 outer contact surface
43 ring collar
44 fastening loop
45 base flange
46 base plate
50 second coupling part
51 axial lock flange
52 inner contact surface
53 axial seal-supporting flange
54 inner seal-supporting surface
55 bulge sealing
56 detachment grip
57 axially extending cut
58 local protrusion
59 inner V-shaped lock flange corner
60 locking ring
61 locking ring clamping mechanism
62 ring groove
63 bag-connection flange
64 proximal flange
70 first inner mould assembly
71 second inner mould assembly
72 first inner mould part
73 second inner mould part
74 third inner mould part
75 intermediate coupling part
76 inner undercut
90 outer contact surface diameter
91 outer ring collar diameter
92 inner contact surface diameter
93 inner seal-supporting surface diameter
94 inner bulge sealing diameter
95 central axis
S1-S3 method steps

1. An ostomy appliance comprising:
a first ring-shaped coupling part comprising an axial upright forming an outer contact surface having an outer contact surface diameter, the axial upright further forming a ring collar having an outer ring collar diameter exceeding the outer contact surface diameter;
a second ring-shaped coupling part comprising an axial lock flange forming an inner contact surface having an inner contact surface diameter less than the outer ring collar diameter, the second coupling part comprising an axial seal-supporting flange axially coextending the axial lock flange and forming an inner seal-supporting surface having an inner seal-supporting surface diameter exceeding the inner contact surface diameter, the second coupling part comprising a bulge sealing extending radially inward from the seal-supporting surface and terminating inwardly at an inner bulge sealing diameter less than the outer ring collar diameter;
and a collecting bag in connection with the second coupling part;
wherein the bulge sealing has a bulge sealing hardness and the ring collar has a ring collar hardness, wherein the bulge sealing hardness is less than the ring collar hardness, and wherein the first coupling part and second coupling part are adapted to be detachably attached to each other in a coupled configuration such that the bulge sealing is positioned against the ring collar to provide a seal between the first coupling part and the second coupling part.

2. The ostomy appliance according to claim 1, wherein the outer contact surface radially contacts the inner contact surface when the first coupling part and the second coupling part are attached to each other in the coupled configuration.

3. The ostomy appliance according to claim 1, wherein the axial lock flange has an axial lock flange hardness, wherein the bulge sealing hardness is less than the axial lock flange hardness.

4. The ostomy appliance according to claim 1, wherein the bulge sealing hardness is based on a material hardness of the bulge sealing, wherein the material hardness is in a hardness range from 40 to 55 when measured according to an ASTM D2240-00 standard on a type OO scale.

5. The ostomy appliance according to claim 1, wherein the bulge sealing is made from a thermoplastic elastomer.

6. The ostomy appliance according to claim 1, wherein the axial lock flange, the axial upright, the seal-supporting flange, or any combination thereof comprise a low-density polyethylene.

7. The ostomy appliance according to claim 1, wherein the ring collar comprises a polyolefin elastomer.

8. The ostomy appliance according to claim 1, wherein the second coupling part comprises a radially extending bag-connection flange axially positioned between the axial lock flange and the seal-supporting flange, and a collecting bag is connected to second coupling part via the bag-connection flange.

9. The ostomy appliance according to claim 8, wherein the ostomy appliance further comprises a locking ring having an open state and a locked state, wherein the second coupling part comprises a radially extending proximal flange connected to the axial lock flange at a distal end relatively to the seal-supporting flange such that the bag-connection flange, the axial lock flange, and the proximal flange collectively form a ring groove for housing the locking ring, wherein the locking ring locks the second coupling part to the first coupling part when the first coupling part and the second coupling part are attached to each other in the coupled configuration and when the locking ring is housed in the ring groove in the locked state.

10. The ostomy appliance according to claim 1, wherein the first coupling part comprises a radially extending base flange connected to the axial upright at a distal end relatively to the ring collar, wherein the base flange axially contacts the second coupling part when the first coupling part and the second coupling part are attached to each other in the coupled configuration.

11. The ostomy appliance according to claim 1, wherein the second coupling part comprises one or more local protrusions extending radially outwardly from the axial seal-supporting flange locally around the second ring-shaped coupling part and extending axially from the bag-connection flange.

12. The ostomy appliance according to claim 1, wherein the inner seal-supporting surface is radially outwardly offset from the outer ring collar diameter.

13. The ostomy appliance according to claim 1, wherein the bulge sealing axially extends across an outermost part of the ring collar when the first coupling part and the second coupling part are attached to each other in the coupled configuration.

14. The ostomy appliance according to claim 1, wherein the bulge sealing forms an inner undercut of the second coupling part.

15. The ostomy appliance according to claim 1, wherein an entire inner seal-supporting surface is outwardly offset from the inner contact surface of the axial lock flange.

16. The ostomy appliance according to claim 1, wherein the inner seal-supporting surface has a non-decreasing diameter along its entire axial direction away from the axial lock flange.

17. The ostomy appliance according to claim 1, wherein the inner bulge sealing diameter is less than the inner contact surface diameter.

18. The ostomy appliance according to claim 1, wherein the lock flange comprises one or more axially extending cuts.

19. The ostomy device according to claim 1, wherein the base flange has a base flange hardness which is less than the ring collar hardness.

20. The ostomy device according to claim 1, wherein the lock flange terminates axially at an inner V-shaped lock flange corner located at an end of the lock flange closest to the bulge sealing, wherein the lock flange corner engages with the ring collar when the first coupling part and the second coupling part are in the coupled configuration.

21. (canceled)

22. (canceled)

23. (canceled)

24. (canceled)

25. (canceled)

26. (canceled)

27. A ring-shaped coupling part for an ostomy appliance, the coupling part comprising:
an axial lock flange forming an inner contact surface having an inner contact surface diameter;
an axial seal-supporting flange axially coextending the axial lock flange and forming an inner seal-supporting surface having an inner seal-supporting surface diameter exceeding the inner contact surface diameter; and
a bulge sealing extending radially inward from the seal-supporting surface and terminating inwardly at an inner bulge sealing diameter less than the outer ring collar diameter;
wherein the bulge sealing has a bulge sealing hardness and the axial lock flange has an axial lock flange hardness, wherein the bulge sealing hardness is less than the axial lock flange hardness.

28. (canceled)

29. (canceled)
